(19) United States
(12) Patent Application Publication
(10) Pub. No.: US 2017/0165293 A1
(43) Pub. Date: Jun. 15, 2017

(54) METHOD AND APPARATUS FOR SCAVENGING PLASMA FREE HEMOGLOBIN

(71) Applicant: GENO LLC, COCOA, FL (US)

(72) Inventors: Kurt A. Dasse, Needham, MA (US); Priscilla C. Petit, Orlando, FL (US); Mark K. Wedel, Temecula, CA (US); David H. Fine, Cocoa Beach, FL (US)

(73) Assignee: GENO LLC, COCOA, FL (US)

(21) Appl. No.: 15/375,108

(22) Filed: Dec. 11, 2016

Related U.S. Application Data
(60) Provisional application No. 62/265,923, filed on Dec. 10, 2015.

(51) Int. Cl. A61K 33/00; A61K 9/00; A61K 38/28; A61K 47/26; A61M 1/16; A61K 33/06; A61K 45/06; A61M 16/08; A61M 1/02; A61M 1/36; A61K 35/14; A61K 31/137

(52) U.S. Cl. CPC A61K 33/00; A61K 35/14; A61K 9/007; A61K 38/28; A61K 47/26; A61K 31/137; A61K 33/06; A61K 45/06; A61M 16/08; A61M 1/02; A61M 1/3666; A61M 1/1698

(57) ABSTRACT

A method of improving hemodynamics includes identifying a mammal having or at risk of developing a vascular depletion of nitric oxide due to nitric oxide scavenging by oxyhemoglobin, and introducing nitric oxide into the mammal's circulation.

This application claims priority under 35 U.S.C. §119(e) to U.S. Patent Application Ser. No. 62/265,923 filed on Dec. 10, 2015, which is hereby incorporated by reference in its entirety.

### TECHNICAL FIELD

The invention relates to administering nitric oxide in a therapeutic setting.

### BACKGROUND

An antioxidant is a molecule that inhibits the oxidation of other molecules. Oxidation is a chemical reaction involving the loss of electrons or an increase in oxidation state. Oxidation reactions can produce free radicals. In turn, these radicals can start chain reactions. When the chain reaction occurs in a cell, it can cause damage or death to the cell. Antioxidants terminate these chain reactions by removing free radical intermediates, and inhibit other oxidation reactions. They do this by being oxidized themselves, so antioxidants are often reducing agents such as thiols, ascorbic acid (vitamin C), or polyphenols.

Nitric oxide, also known as nitrosyl radical, is a free radical that is an important signalling molecule. For example, NO can cause smooth muscles in blood vessels to relax, thereby resulting in vasodilation and increased blood flow through the blood vessel. These effects can be limited to small biological regions since NO can be highly reactive with a lifetime of a few seconds and can be quickly metabolized in the body.

In hemolytic diseases, cell-free hemoglobin (Hb) has been postulated to be responsible for impaired endothelial function and pathogenic abnormalities of the vasculature. Proposed mechanisms have been based on nitric oxide (NO) scavenging by oxyhemoglobin (oxyHb) or processes mediated by oxidative reactions of methemoglobin (metHb). However, there has been uncertainty surrounding the relationship between Hb decompartmentalization and NO consumption. Indeed, a role for Hb in mediating NO scavenging and for the erythrocyte in limiting this process has been challenged on conceptual grounds. In addition, the primary mechanism for the vasoreactivity of cell-free Hb-based blood substitutes remains controversial and has been uncertain, attributed to both NO scavenging and the premature delivery of oxygen to the systemic arterioles both of which mediate vasoconstriction.

### SUMMARY

The claimed method of improving hemodynamics includes identifying a mammal having or at risk of developing a vascular depletion of nitric oxide due to nitric oxide scavenging by oxyhemoglobin, positioning a mammal, such as a patient, for nitric oxide treatment, administering nitric oxide for aiding conversion of oxyhemoglobin to methemoglobin, preventing scavenging effects of oxyhemoglobin, and introducing the nitric oxide into the circulation.

Examples of such conditions can include cardiac injury, hepatic injury, pulmonary injury, preeclampsia and hemolysis or a combination of any of these injuries. Patients can be neonates, pediatric patients, or adults.

The mammal can be treated with a sedative or an analgesic or both, and oxygen saturation levels can be monitored. The nitric oxide can be inhaled nitric oxide, which may be administered by introducing it into a respiratory breathing circuit.

The inhaled nitric oxide can be administered in an amount effective to prevent systemic vasoconstriction.

The nitric oxide can be administered up to 80 ppm, but more typically in the 5 to 20 ppm range, and sometimes as low as 0.1 to 1.0 ppm, depending upon the circumstances.

The nitric oxide can be administered before, during and/or after a first transfusion.

The method can be a transfusion, and the transfusion can be an exchange transfusion.

The method can further include delivering a hydrogen gas.

The hydrogen can actsto eliminate peroxynitrite, thereby reducing adverse effects of nitric oxide.

The method can further include delivering a subsequent transfusion.

The method can further include comprising culturing red blood cells to detect contamination prior to transfusion.

The method can include administering nitric oxide before, during and/or after a first transfusion.

The concentration of nitric oxide in the gas mixture delivered is at least 0.1 ppm, and in some embodiments, and up to 5 ppm for the desired effect. In certain embodiments, the nitric oxide can also be titrated up to 80 ppm should a higher dose be required.

In other embodiments, nitric oxide can be administered up to 0.08 ppm, up to 0.8 ppm, or up to 8 ppm.

In certain embodiments, the method can include exchanging 65 to 85 percent blood volume over a period of 2-12 hours for preemies and term babies, and the method can assume that the estimated circulating blood volume is 80 ml/kg for term babies, and 100 ml/kg for term babies.

The transfusion can include exchanging the same percent of blood volume of the same period of time.

In some embodiments, the method can further include monitoring calcium (Ca) levels in the mammal during transfusion, and if Ca<0.7 mEq, providing an emergency treatment for hypocalcemia of 10 ml CaCl in 50-100 ml D5W given IV over 5 to 10 minutes.

The method can further include monitoring potassium levels in the mammal during transfusion, and if K>6.5, administering 10-15 units IV of regular insulin along with 50 ml D50W, plus/minus 10-20 mg salbutamol by nebulization, and calcium (see dose below) in the presence of malignant cardiac arrhythmias.

The method can further include administering analgesia.

In the claimed method, the level of anesthesia can be evaluatedcontinuously.

The transfusion can involve using stored blood, greater than 7 days old.

The transfusion can involves using fresh blood, no more than 7 days old.

In certain embodiments, hydrogen gas can be combined with the nitric oxide in a breathing gas.

In other embodiments, nitric oxide is provided in an amount effective to minimize acute renal injury.

In certain embodiments, the nitric oxide is provided in an amount effective to minimize loss of the neuroprotective effect in the brain.

In some embodiments, nitric oxide is provided in an amount effective to minimize loss of the protective effect in the lungs.

In some examples, the nitric oxide is provided in an amount effective to minimize loss of the protective effect in the heart.

In some examples, the nitric oxide is provided in an amount effective to minimize loss of the protective effect in the liver.

In certain embodiments, wherein the nitric oxide is provided in an amount effective to minimize loss of the protective effect during cardiac injury, hepatic injury, pulmonary injury, or a combination of any of these injuries.

In yet other embodiments, the nitric oxide is provided in an amount effective to minimize loss of the protective effect during preeclampsia and hemolysis.

In yet other embodiments, the nitric oxide is provided in an amount effective to minimize hemolysis during sepsis.

In other examples, the nitric oxide is provided in an amount effective to minimize loss of the protective effect during disseminated intravascular coagulopathy (DIC).

In yet other examples, the nitric oxide is provided in an amount effective to minimize loss of the protective effect during transplantation, organ preservation, during support with mechanical circulatory support devices including left, right, and biventricular assistance and extracorporeal membrane oxygenation (ECMO), and during cardiopulmonary bypass procedures.

In some embodiments, the nitric oxide is administered to neonates, to pediatric patients, or to adults, or any combination of each.

In yet other examples, the nitric oxide is provided in an amount effective to minimize loss of the protective effect during sickle cell anemia, in the presence of a mechanical and/or malfunctioning native valve, or for neonates with hemolytic anemia with persistent pulmonary hypertension of the newborn (PPHN).

The method can be applied to any condition leading to elevated circulating cell-free hemoglobin due to acute or chronic hemolysis. Hemolysis is defined as cell free hemoglobin exceeding 5 mg/dl and/or a reduction in haptoglobin with or without a concomitant increase in reticulocyte count.

A system for improving hemodynamics can include a table for positioning a mammal to receive nitric oxide treatment, a monitor configured to detect oxygen saturation levels, a device for administering nitric oxide in an amount and frequency effective to convert oxyhemoglobin to methemoglobin in the mammal's circulation and prevent scavenging effects of oxyhemoglobin.

The system can further include a sedation source. The sedation source can include anesthesia.

The system can further include an analgesia source.

The system can include a cartridge to convert nitric oxide-releasing agents to NO. The cartridge can include an inlet, an outlet, and a reducing agent. The cartridge can be configured to utilize the whole surface area in converting nitric oxide-releasing agents to NO. The cartridge can have a length, width, and thickness, an outer surface, and an inner surface, and can be substantially cylindrical in shape. The cartridge can have aspect ratio of approximately 2:1, 3:1 or 4:1. The length can be, for example, one inch, two inches, three inches, four inches or five inches. The width can be, for example, 0.5 inch, 1 inch, 1.5 inches, 2 inches, or 2.5 inches. The cartridge can have a cross-section that is a circle, oval, or ellipse. In certain embodiments, opposing sides along the length of the cartridge can be flat. The thickness between the inner and outer surface can be constant, thereby providing a uniform exposure to the reducing agents. The thickness can be approximately 1 mm, 2 mm, 5 mm, 10 mm, 20 mm, 30 mm, or 40 mm for example.

Other features, objects, and advantages will be apparent from the description, drawings, and claims.

### BRIEF DESCRIPTION OF THE DRAWINGS

FIG. 1 is a schematic showing an embodiment of the claimed method.

FIG. 2 depicts the mortality associated with transfusion of older blood compared to newer blood.

FIG. 3 shows the effects of hemolysis and inhaled NO on mean arterial pressure (MAP).

FIG. 4 shows the relationship between total cell-free plasma Hb and the physiologic effects of hemolysis and inhaled NO.

FIG. 5 shows effects of hemolysis and inhaled NO on renal function

FIG. 6 shows plasma NO consumption and plasma Hb levels.

FIG. 7 shows the effects of sodium nitroprusside during hemolysis with and without inhaled NO.

FIG. 8 shows the effects of Hb infusions with and without inhaled NO.

FIG. 9 shows changes in hemodynamic values.

FIG. 10 shows NO consumption ability.

FIG. 11 shows Hb species and percent changes in MAP during oxyHb Infusions.

FIG. 12 shows a cartridge that can be applied in the claimed methods.

FIG. 13 shows various components used with a cartridge.

### DETAILED DESCRIPTION

Nitric oxide is an important signaling molecule in pulmonary vessels. Nitric oxide can moderate pulmonary hypertension caused by elevation of the pulmonary arterial pressure. Inhaling low concentrations of nitric oxide, for example, in the range of 0.1-80 ppm can rapidly and safely decrease pulmonary hypertension in a mammal by vasodilation of pulmonary vessels.

NO has been shown to prevent vasoconstriction observed at similar levels of oxyhemoglobin (e.g., 200 uM) in a model of hemolysis. Applicants' data supports that NO's ability to prevent vasoconstriction is due to the NO aiding in the conversion of oxyhemoglobin to methemoglobin thereby preventing the scavenging effects of oxyhemoglobin producing pulmonary hypertension and tissue damage thereby improving survival associated with an exchange transfusion of older stored blood in the critically ill mammals, e.g., canines with pneumonia or renal failure.

In a well-established sedated and ventilated canine model of pneumonia-induced sepsis treated with standard hemodynamic support, the inventors proposed to perform a massive exchange transfusion of older (42 day old) stored blood and fresh (7 day old) blood in all animals and randomize animals to receive inhaled NO or to not receive inhaled NO and compare outcomes including survival over 96 hours.

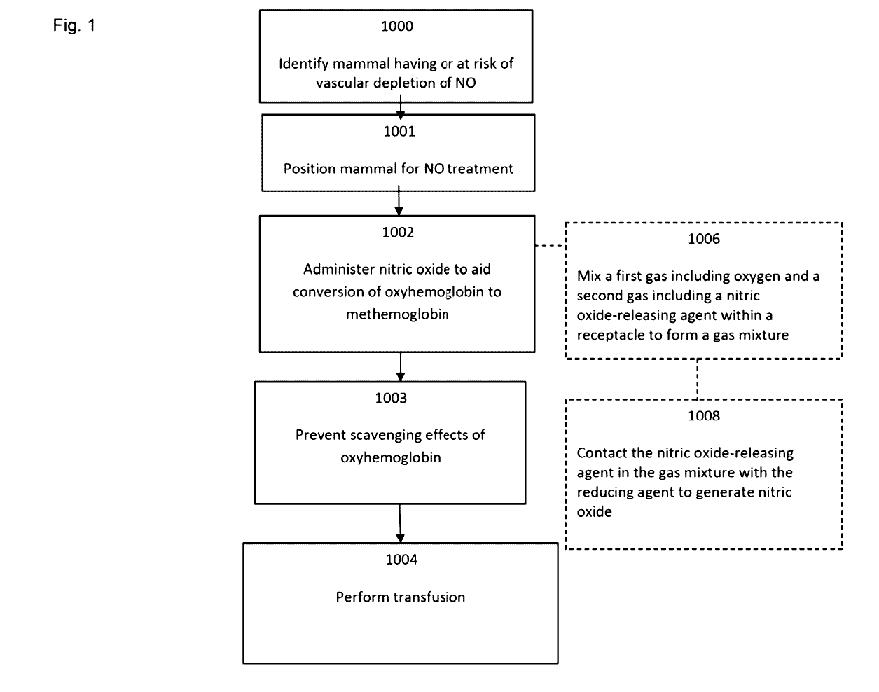

Referring to FIG. 1, the claimed method of improving hemodynamics can include identifying a mammal having or at risk of developing a vascular depletion of nitric oxide due to nitric oxide scavenging by oxyhemoglobin (1000), positioning a mammal for nitric oxide treatment (1001), administering nitric oxide for aiding conversion of oxyhemoglobin to methemoglobin (1002), preventing scavenging effects of oxyhemoglobin (1003), and performing a transfusion (1004). Identifying such a mammal having or at risk of developing a vascular depletion of nitric oxide due to nitric oxide scavenging by oxyhemoglobin typically includes making a diagnosis based on a physical examination including vital signs, laboratory tests (e.g. blood work, complete blood count (CBC), and metabolic panel including potassium and calcium levels) and ancillary testing (e.g., imaging studies for example). This typically further involves planning a course of treatment, communicating the diagnosis and treatment plan, and preparing the mammal for treatment.

The mammal can be treated with a sedative or an analgesic or both, and oxygen saturation levels can be monitored. The nitric oxide can be inhaled nitric oxide, which may be administered by introducing it into a respiratory breathing circuit. The nitric oxide can be provided in an amount and manner effective to minimize acute renal injury. The nitric oxide can be administered in an amount and manner effective to prevent systemic vasoconstriction.

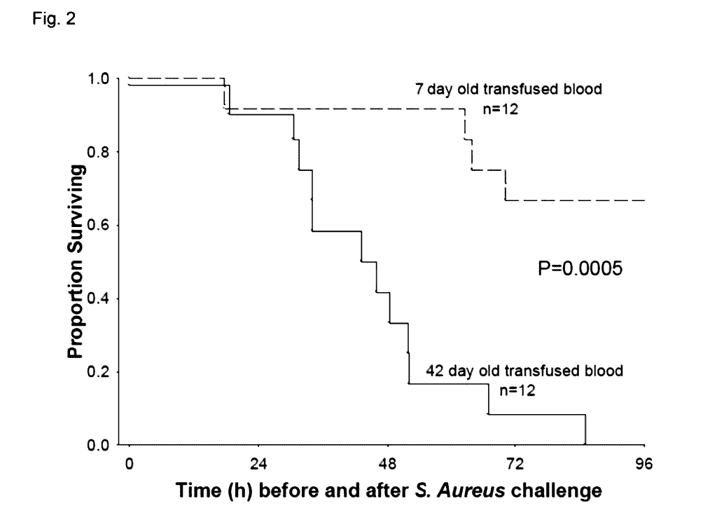

Referring to FIG. 2, it has been shown in a meta-analysis of blood transfusion studies including critically ill patients that receiving transfused “older” stored blood resulted in a significantly higher mortality as compared to “newer” stored blood. Data shows that the mortality associated with exchange transfusion of 42 day old (limit of the FDA approved storage period for canines and humans) versus 7 day old stored blood in canines with pneumonia is significantly increased. This increase in mortality is believed to be due to in vivo hemolysis with the release of vasoactive cell-free oxyhemoglobin over days after transfusion. The oxyhemoglobin scavenges nitric oxide (NO), an endogenous vascular vasodilator resulting in acute pulmonary arterial hypertension, compromise of cardiac function, and pulmonary tissue damage (necrosis and hemorrhage) at the site of infection.

In one study, as shown in Table 1 below, a full-factorial study design was performed to study of the effects of intravascular hemolysis and inhaled NO. See, e.g., Minneci, et al., Hemolysis-associated endothelial dysfunction mediated by accelerated NO inactivation by decompartmentalized oxyhemoglobin, J. Clin. Investigation, December 2005, which is incorporated by reference herein. In order to minimize variability and limit the number of animals necessary to perform these studies, each animal underwent a baseline and intervention experiment. During the first week, each animal underwent a 6-hour baseline study with an infusion of D5W to control for the effects of the fluid challenge. During the second week, animals underwent a 6-hour intervention study in which they were randomized to receive 1 of 4 treatments (D5W; D5W plus inhaled NO; free water; or free water plus inhaled NO). This design allowed for the comparison of differences across treatment groups by subtracting calculated differences within animals (from baseline to intervention) in each treatment group. Comparison of these differences of the differences allowed for analysis of the effects of hemolysis, the effects of inhaled NO, and detection of any interaction between the 2 interventions.

TABLE 1

| N = 20 | Week 1 - Baseline | | Week 2 - Intervention |
|---|---|---|---|
| | D5W infusion | — | D5W infusion |
| | D5W infusion | — | D5W infusion |
| | D5W infusion | — | Water infusion + inhaled NO |
| | D5W infusion | — | Water infusion + inhaled NO |

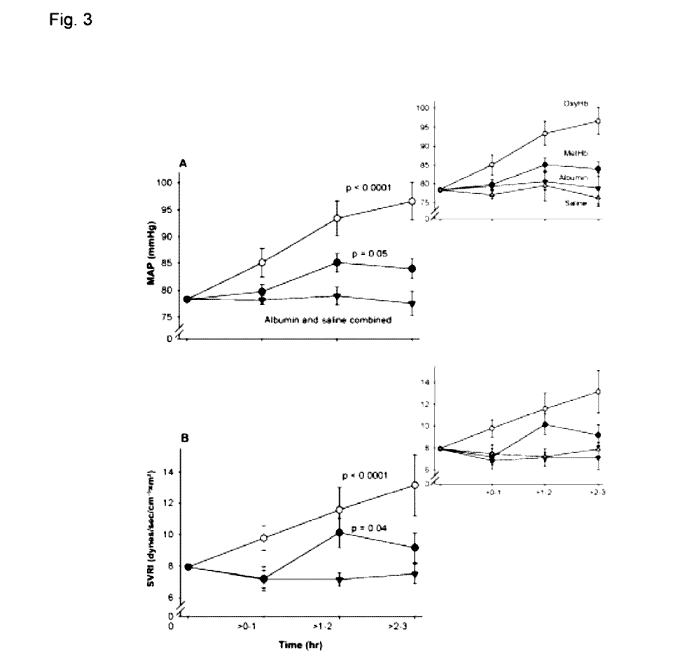

Referring to FIG. 3, this graph shows the effect of hemolysis and inhaled NO on MAP. In paired experiments, all animals has received a 6-hour D5W infusion during the baseline study and 1 week later were randomized to a 6-hour intervention study of either D5W, D5W plus NO, free water, or free water plus NO. Changes in MAP over the course of the 6-hour baseline (filled circles) and intervention studies (open circles) are shown. In all 4 groups of animals, there were statistically similar small increases in MAP during the 6-hour baseline D5W infusion. Compared with an equivalent infusion of D5W with or without NO (non-hemolyzing control groups), free water-induced intravascular hemolysis caused a significant increase in MAP, which was attenuated by the concurrent inhalation of NO gas (P=0.0003 for interaction of NO and hemolysis). See Minneci, 2005.

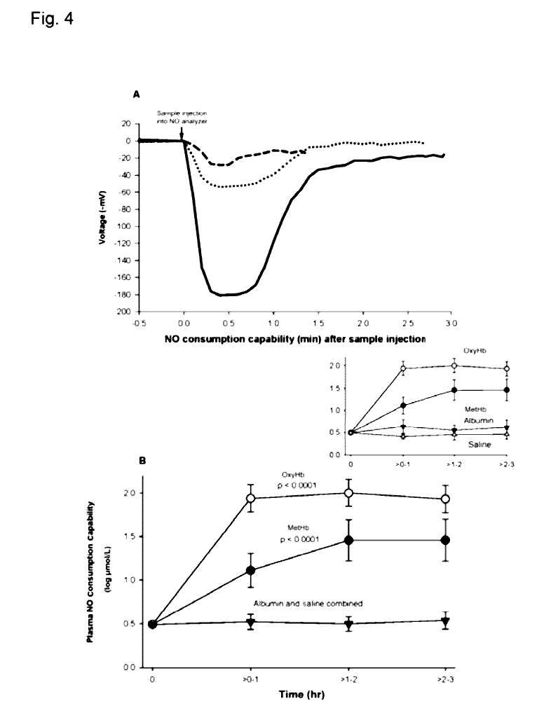

Referring to FIG. 4, this shows the relationship between total cell-free plasma Hb and the physiologic effects of hemolysis and inhaled NO. In the Upper panels, this shows the difference in response from 0 to 6 hours between baseline and intervention studies for each of the 4 treatment groups is shown for MAP and SVRI. In animals receiving D5W (non-hemolyzing control groups), inhaled NO had no net effect on MAP and SVRI. Compared with these non-hemolyzing controls, free water-induced intravascular hemolysis caused significant increases in MAP and SVRI, which were attenuated by the concurrent inhalation of NO gas (P=0.0003 for interaction of NO and hemolysis for both variables). In the Lower panels: this shows the relationship between change in MAP and SVRI and total plasma Hb levels (concentration in terms of heme groups) during the intervention studies in the hemolyzing groups (free water and free water plus NO groups). Despite similar total plasma Hb levels in these 2 groups, the relationships between change in MAP and SVRI and total plasma Hb levels were significantly different (P=0.003 and P=0.001, respectively). As total plasma Hb levels increased, MAP and SVRI increased more in the free water group than in the free water plus NO group. See id.

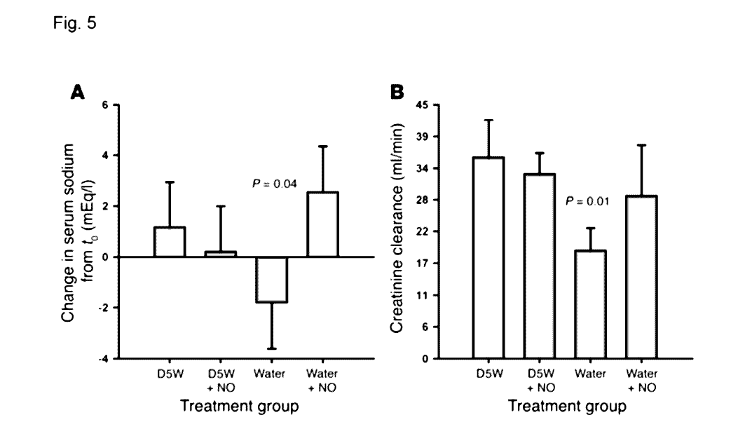

Referring to FIG. 5, this shows the effects of hemolysis and inhaled NO on renal function. (A) The difference in response from 0 to 6 hours between baseline and intervention studies for each of the 4 treatment groups is shown for serum sodium levels. Compared with infusion of D5W with or without NO, free water-induced intravascular hemolysis caused a significant impairment in the ability of the kidneys to compensate for hyponatremia, which was attenuated by the concurrent inhalation of NO (P=0.04). (B) Six-hour creatinine clearance values during the intervention studies for each of the 4 treatment groups are shown. Based on a priori hypotheses, the creatinine clearance values ordered as expected, with the free water group having the lowest clearance, the D5W and D5W plus NO groups having the highest clearances, and the free water plus NO group having an intermediate clearance approaching that of the D5W and D5W plus NO groups (P=0.01). See id.

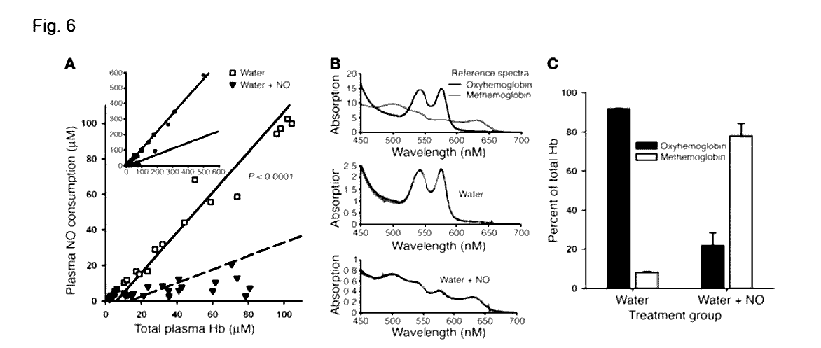

Referring to FIG. 6, this shows the plasma NO consumption and plasma Hb levels. FIG. 6 (A) shows that a significantly different relationship exists between plasma NO consumption and total plasma Hb levels (concentration in terms of heme groups) in the free water and free water plus NO groups (P<0.0001). The inset demonstrates the relationships over the entire range of measured Hb levels, whereas the main graph focuses on the physiologic range of hemolysis in human disease states. FIG. 6(B) shows spectral deconvolution of the plasma Hb species. The upper spectrum represents reference tracings for canine oxyhemoglobin and methemoglobin. The middle and lower spectra represent characteristic samples from the free water and free water plus NO treatment groups, respectively. FIG. 6(C) shows total plasma Hb composition in the free water and free water plus NO groups was significantly different at 6 hours (P=0.03). In the free water group, the plasma contained predominantly oxyhemoglobin. In contrast, in the free water plus NO group, the plasma contained predominantly methemoglobin. See id.

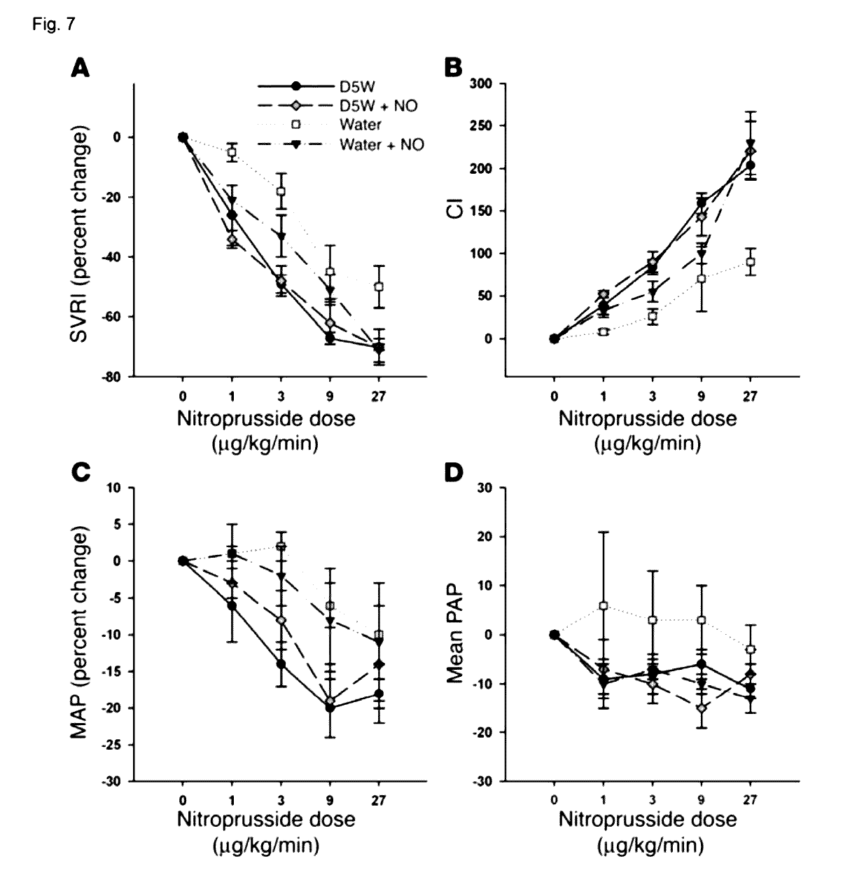

Referring to FIG. 7, this shows the physiologic effects of sodium nitroprusside during hemolysis with and without inhaled NO. Percent change in SVRI (A) and CI (B) in response to increasing doses of sodium nitroprusside during the intervention studies for each of the 4 treatment groups. Compared with D5W and D5W plus NO, free water-induced hemolysis led to blunted hemodynamic effects of escalating doses of sodium nitroprusside, which were restored with inhaled NO therapy and oxidation of plasma Hb (P=0.005 and P=0.02 for SVRI and CI, respectively). Similar but not statistically significant patterns of response to increasing doses of sodium nitroprusside in the 4 treatment groups were also demonstrated for MAP (C), PAP (D), heart rate, CVP, and PCWP. In fact, all 7 hemodynamic variables demonstrated the expected ordered responses to nitroprusside (P=0.008 for 7/7 variables having the same response pattern). See id.

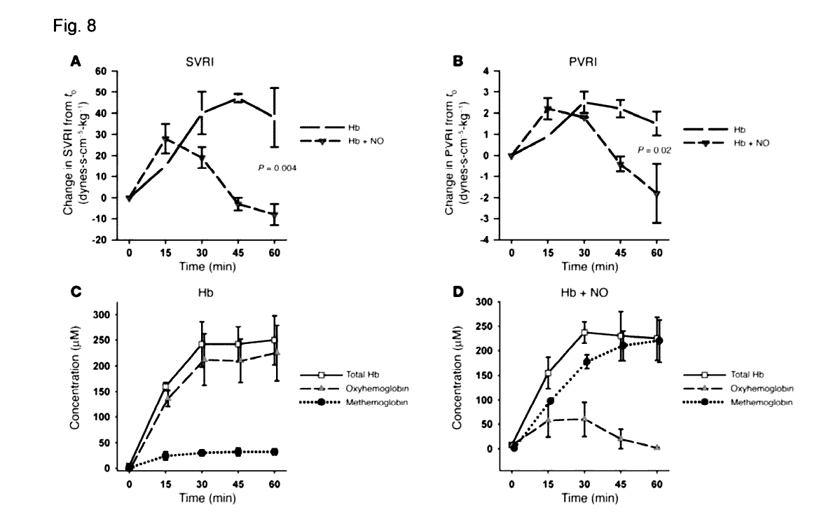

Referring to FIG. 8, this shows the effects of Hb infusions with and without inhaled NO. Infusion of cell-free Hb led to increases in SVRI and pulmonary vascular resistance index that were attenuated by inhaled NO (A and B). In animals breathing air (n=2), the cell-free Hb remained predominantly oxyhemoglobin (C). In contrast, in animals breathing NO (n=2), the cell-free Hb was converted to methemoglobin (D). See id.

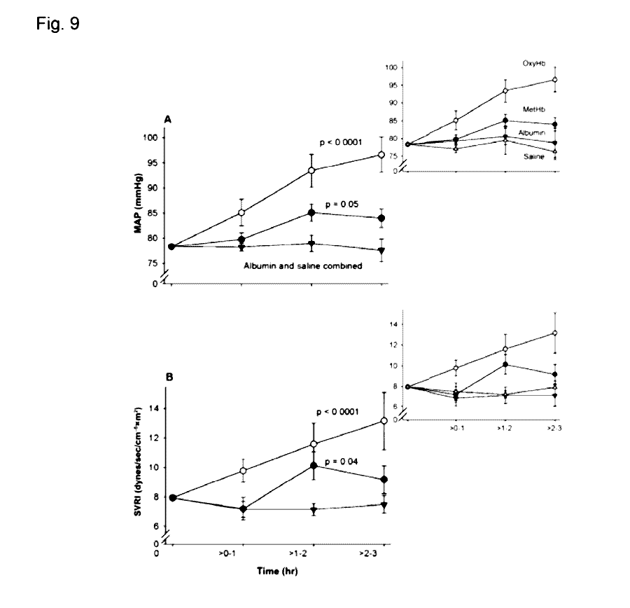

Referring to FIG. 9, this shows changes in MAP and SVRI. Serial mean (SE) changes in (A) MAP and (B) SVRI in animals receiving oxyHb (n=5), metHb (n=5), albumin (n=5), or saline (n=5) are plotted. Hemodynamic values are plotted from a common origin representing the mean values for all animals at Time 0. The inset above and to the right shows the individual serial changes for albumin and saline controls compared to the other two treatment groups. p value represents changes over time compared to the combined controls. See, e.g., Wang, D. et al., In vivo reduction of cell-free methemoglobin to oxyhemoglobin results in vasoconstriction in canines, Transfusion 53, p. 3149-3163 (December 2013), which is incorporated by reference herein. FIG. 9 shows the time course of vascular pressure changes of the four study groups. The albumin and saline groups were combined since they are similar. After the cell-free oxyHb (Fe2+-O2) infusion was completed (0-1 hr), there were until the end of the experiment (1-3 hr) significant elevations in mean MAP (p<0.0001) and SVRI (p<0.0001) compared to controls (albumin and saline). Unexpectedly, after cell-free ferric metHb (Fe3+) infusions, there were also over this time period elevations in MAP (p=0.05) and mean SVRI (p=0.04) compared to control animals. However, despite infusing similar concentrations of Hb solutions over 1 hour, the metHb infusions produced significantly less of an increase in MAP and SVRI compared to the oxyHb infusions (p=0.006 and p=0.04, respectively).

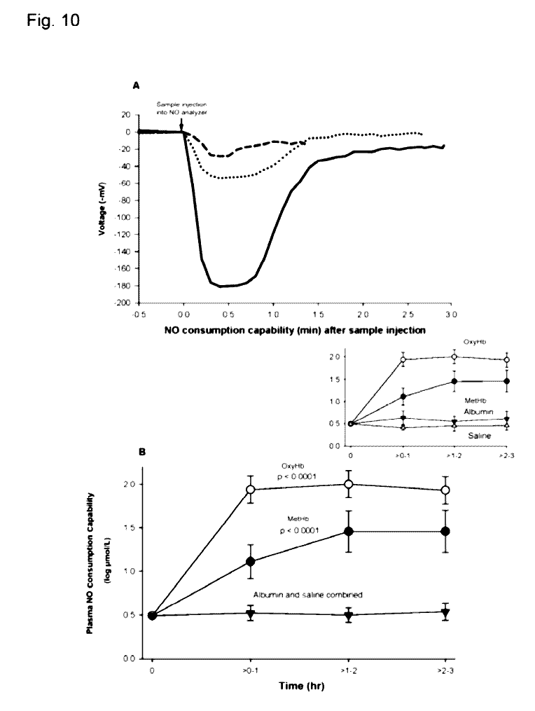

Referring to FIG. 10, this shows NO consumption. FIG. 10(A) shows plasma NO consumption capability obtained from animals 1 hour after infusion of various Hb species or albumin. FIG. 10 (B) shows a format similar to FIG. 9, except that the mean (+/−SE) log NO consumption capability of plasma is plotted. -, oxyHb group; • • •, metHb group; - - -, albumin group. This assay uses the fact that oxyHb is a very potent NO scavenger and that presence of any traces of oxyHb in plasma will result in loss of plasma NO. In practice, a chemiluminescence NO detector is used to measure changes in the steady state NO in a bath with a NO donor present. If, with the addition of plasma, NO is scavenged, the steady-state level of NO decreases, which is observed as a decrease in voltage in the detector of the NO analyzer (FIG. 10A). This voltage decrease indicates the presence of cell-free oxyHb (or potentially other NO-scavenging species such as ceruloplasmin) in plasma. Elevated NO consumption ability of plasma in samples collected after oxyHb and metHb infusions compared to controls with infused albumin and saline (both p<0.0001; FIG. 10B). Increase in NO consumption ability of plasma was highest with oxyHb. Unexpectedly, plasma from metHb infusions was also able to consume NO, albeit at a 10-fold lower level than the infused oxyHb-containing plasma (p=0.009; FIG. 10B), consistent with the decreased vasoconstrictive properties associated with metHb infusions.

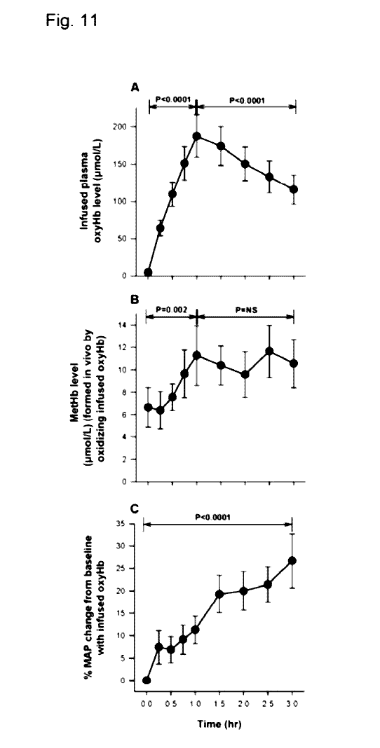

Referring to FIG. 11, this shows Hb species and percent changes in MAP during oxyHb infusions. FIG. 11 (A) shows serial mean (+/−SE) values of oxyHb levels. FIG. 11 (B) shows serial mean (+/−SE) metHb levels formed by oxidizing a fraction of the oxyHb infusion in vivo. FIG. 11 (C) shows mean (+/−SE) percent increase in MAP during the oxyHb infusion. All p values compare changes over the time period indicated by brackets.

FIG. 11A shows oxyHb levels in plasma as a function of time—levels increased progressively during the 1-hour infusion (p<0.0001 for slope) and then monotonically decreased over the 2 hours after the infusion stops (p<0.0001 for slope). The concentration of cell-free oxyHb oxidized in plasma to metHb is plotted in FIG. 11B and the levels of metHb progressively increased during the 1-hour oxyHb infusion (p=0.002 for slope) and remained elevated and unchanged during the past 2 hours of the experiment. FIG. 11C shows the MAP similarly increasing throughout the 3-hour experiment (27% increase from 0 to 3 hr, p<0.0001).

Studies have determined during infusion there was overall a significant positive relationship between increasing oxyHb levels and increases in MAP (p=0.03 for slope; FIG. 4A, left side). Moreover, during infusion, there was at each time point studied a similar positive correlation between increases in MAP and oxyHb plasma levels (0.25, 0.50. 0.75, and 1.0 hr; r=+0.79 to +0.91; FIG. 4A, right side). This strong positive correlation during the infusion occurred over a wide range of oxyHb values; near the start of the infusion (0.25 hr), plasma concentrations in the five animals studied ranged from approximately 40 to 90 mmol/L, and by the end of the infusion (1 hr), they varied from approximately 90 to 250 mmol/L. However, once the oxyHb infusion ended and oxyHb levels fell, the correlations between oxyHb plasma levels and increases in MAP at each time point measured became weaker (r=+0.80 to −0.06). In summary, the relationship between oxyHb plasma levels and MAP was very strong during infusion of oxyHb over a wide range of plasma levels. After the infusion ended and levels were decreasing, likely in part because the elimination of oxyHb from plasma became more prominent, the correlation progressively weakened and became overall nonsignificant. In contrast, metHb levels converted from infused oxyHb (product of oxyHb oxidation in plasma) were not correlated with changes in MAP throughout the experiment.

### Neuroprotective Properties of NO in the Brain

Circulating NO serves as a signalling molecule that induces a neuroprotective effect in the brain during hypoxia and oxidative stress. Likewise, inhaled NO bonds to haemoglobin and is transported to the brain. This has been shown to provide the same neuroprotection during oxidative stress. A decrease in endogenous NO would induce a loss of this protection. The harmful effects of elevated cell-free haemoglobin due to scavenging endogenous NO could be compensated for by supplemental delivery of exogenous NO. A measure of compensation will be demonstrated through improvement of cognition when exogenous NO is provided after induction of hemolysis compared to untreated controls. Biomarkers of oxidative stress will also be measured and shown to decrease with the addition of inhaled NO.

### Protective Properties of NO in the Lungs

Circulating NO serves as a molecule that induces a protective effect in the lungs during hypoxia and oxidative stress. Likewise, inhaled NO bonds to haemoglobin when delivered through the lungs. This has been shown to provide protection during oxidative stress. A decrease in endogenous NO would induce a loss of this protection. The harmful effects of elevated cell-free haemoglobin due to scavenging endogenous NO could be compensated for by supplemental delivery of exogenous NO. A measure of compensation will be demonstrated through a reduction in vasoconstriction when exogenous NO is provided after induction of hemolysis compared to untreated controls. Biomarkers of oxidative stress will also be measured and shown to decrease with the addition of inhaled NO.

### Protective Properties of NO in the Liver

As discussed above with respect to the lungs, circulating NO serves as a molecule that induces a protective effect in the liver during hypoxia and oxidative stress. Likewise, inhaled NO bonds to haemoglobin and is transported to the liver. This has been shown to provide protection during oxidative stress. A decrease in endogenous NO would induce a loss of this protection. The harmful effects of elevated cell-free haemoglobin due to scavenging endogenous NO could be compensated for by supplemental delivery of exogenous NO. A measure of compensation will be demonstrated by reducing vasoconstriction in the liver when exogenous NO is provided after induction of hemolysis compared to untreated controls. Biomarkers of oxidative stress will also be measured and shown to decrease with the addition of inhaled NO.

### Protective Properties of NO in the Heart

As discussed above with respect to the lungs and liver, circulating NO serves as a molecule that induces a protective effect in the heart during hypoxia and oxidative stress. Likewise, inhaled NO bonds to haemoglobin and is transported to the heart. This has been shown to provide protection during oxidative stress. A decrease in endogenous NO would induce a loss of this protection. The harmful effects of elevated cell-free haemoglobin due to scavenging endogenous NO could be compensated for by supplemental delivery of exogenous NO. A measure of compensation will be demonstrated by reducing vasoconstriction when exogenous NO is provided after induction of hemolysis compared to untreated controls. Biomarkers of oxidative stress will also be measured and shown to decrease with the addition of inhaled NO.

### Protective Properties of NO in Transplants.

NO may provide protection before, during and after an organ transplant by minimizing the onset of oxidative stress. A decrease in endogenous NO would induce a loss of this protection. The harmful effects of elevated cell-free haemoglobin due to scavenging endogenous NO could be compensated for by supplemental delivery of exogenous NO. For this reason, NO can protect donor organs in transplantation. Biomarkers of oxidative stress will also be measured and shown to decrease with the addition of inhaled NO. During transplantation, support devices include left, right, or biventricular assist devices, or any combination of such devices, during extracorporeal membrane oxygenation (ECMO), and cardiopulmonary bypass procedures.

### Protective Properties of NO in Organ Preservation

Similar to the reasons in transplantation, NO can protect donor organs during preservation. This can be in the context of transplantation, or in other contexts, such as when a portion of an organ is excised for clinical or histopathologic examination. Biomarkers of oxidative stress will also be measured and shown to decrease with the addition of inhaled NO. During transplantation, support devices include left, right, or biventricular assist devices, or any combination of such devices, during extracorporeal membrane oxygenation (ECMO), and cardiopulmonary bypass procedures.

### Protective Properties of NO During Sepsis

A reduction in the level of hemolysis during sepsis will be demonstrated during and following sepsis. Biomarkers of oxidative stress will also be measured and shown to decrease with the addition of inhaled NO.

### Plasma Nitrite Levels

To determine if intravascular scavenging of NO by oxyHb (infused or converted) was responsible for the increases in MAP, researchers have measured plasma nitrite levels in animals at several time points. Nitrite can be converted to NO and is also a biomarker for NO production by endothelial NO synthase (eNOS). The mean nitrite levels were similar in animals receiving oxyHb and metHb infusions, compared to controls. Furthermore, in all four treatment groups, nitrite concentration did not significantly change throughout the experiment; the concentrations in plasma ranged on average from approximately 120 to 250 nmol/L throughout (all, p>0.05). Wang, D. et al., p. 3159.

If a NO deficit in the luminal space of the vasculature is causing increases in MAP, then these two variables should be strongly correlated. As expected, researchers found that there was a strong correlation between the level of oxyHb in plasma and MAP levels during the 1-hour oxyHb infusions, measured every 15 minutes over a wide range of gradually increasing plasma levels from 90 to 250 mmol/L (FIG. 4A, top panels). Unexpectedly, after the cell-free oxyHb infusion ended and the plasma oxyHb levels were decreasing over the ensuing 2 hours (but still in the same range, 90-250 mmol/L), the correlation with MAP became nonsignificant. Despite this loss of correlation after the infusion ended, the MAP continued to steadily increase over the next 2 hours at the same rate as during the infusion (FIG. 11C). The loss of correlation over time but continued increase in MAP could be explained in part by the fact that once the infusion stops, elimination of oxyHb from the systemic circulation becomes more prominent and the relationship between NO and oxyHb after this point becomes more complex. However, levels of oxyHb remain high enough vascularly or perivascularly to continue to increase MAP. Based on in vitro experiments, the rate of oxyHb reaction with NO is limited only by diffusion, so any free NO in the plasma will be quickly scavenged in the presence of cell-free oxyHb.

Previous work has suggested that the extent of the effect of intravascular oxyHb on the concentration of NO at the smooth muscle decreases as the concentration of Hb increases to high enough levels. The fact that vascular pressures markedly differ even with very high plasma oxyHb levels in both oxyHb- and metHb-infused animals in excess of plasma NO available to be scavenged can only be explained if the effect of cell-free Hb is not limited to the luminal space and potentially occurs more perivascularly.

In previous experiments (Minneci, 2005) it was shown that inhaled NO, 80 parts per million in canines, which by itself has miniscule or no measurable effects on systemic blood pressure, completely eliminates, by oxidizing oxyHb, the hypertensive effects associated with intravascular free water induced hemolysis and release of cell-free oxyHb. Yu et al. have performed experiments in knockout mice without eNOS (enzyme that produces NO), which are hypertensive compared to wild-type animals. Notably, HBOCs increase vascular pressure in wild-type mice, but completely lose this ability in these eNOS knockout mice.

This study shows that cell-free metHb infusions, after being reduced to oxyHb, increase vascular pressures. Since the cell-free metHb is reduced in vivo in the plasma to oxyHb, and then increases vascular pressure, it is difficult to ascribe these hypertensive effects to RBC membranes or other impurities in the process of formation of ex vivo cell-free Hb. Overall, the above data show that NO scavenging ability of the oxyHb molecule at a minimum is at least responsible for some of the hypertensive vascular effects and potentially the vasculopathies associated with cell-free Hb in various disease states.

It should also be emphasized that cell-free metHb is cleared faster and/or is less stable than cell-free oxyHb in plasma. This is an unexpected finding, as the classic mechanism of Hb clearance is through binding to haptoglobin and subsequent internalization through CD 163 receptor and clearance from plasma by either macrophages or liver hepatocytes. This clearance mechanism is not known to discriminate between oxyHb and metHb. This suggests that clearance is not increased but may be due to metHb dissociating faster favoring formation of dimers and heme dissociating faster from dimers. With a higher percentage of dimers present, this might increase clearance by haptoglobin.

Alternatively, since heme has a different absorption spectra than metHb, this could give the appearance of faster clearance given our use of spectroscopic methods to measure metHb in the plasma.

### Hydrogen Supplement

Hydrogen gas can act as an antioxidant and is a free radical scavenger. Hydrogen is the most abundant chemical element in the universe, but is seldom regarded as a therapeutic agent. Recent evidence has shown that hydrogen is a potent antioxidative, antiapoptotic and anti-inflammatory agent and so may have potential medical applications in cells, tissues and organs.

Using a mixture of NO and hydrogen gases for inhalation can be useful, for example, during planned coronary interventions or for the treatment of ischemia-reperfusion (FR) injury. In short, inhaled NO suppresses the inflammation in FR tissues and hydrogen gas eliminates the adverse by-products of NO exposure, peroxynitrite.

However until applicants' discovery, there has not been a successful combination of hydrogen gas with breathing gas using the claimed apparatus and methods. NO's effect as an antioxidant may be enhanced by eliminating highly reactive by-products of NO inhalation such as peroxynitrite, by adding H2 to inhaled NO gas. Specifically, 1) mice with intratracheal administration of LPS exhibited significant lung injury, which was significantly improved by 2% H₂ and/or 20 ppm NO treatment for 3 hours starting at 5 minutes or 3 hours after LPS administration; 2) H₂ and/or NO treatment inhibited LPS-induced pulmonary early and late NF-κB activation; 3) H₂ and/or NO treatment down-regulated the pulmonary inflammation and cell apoptosis; 4) H₂ and/or NO treatment also significantly attenuated the lung injury in polymicrobial sepsis; and 5) Combination therapy with subthreshold concentrations of H₂ and NO could synergistically attenuate LPS- and polymicrobial sepsis-induced lung injury. In conclusion, these results demonstrate that combination therapy with H₂ and NO could more significantly ameliorate LPS- and polymicrobial sepsis-induced ALI, perhaps by reducing lung inflammation and apoptosis, which may be associated with the decreased NF-κB activity. Studies have shown that hydrogen gas exhibits cytoprotective effects and transcriptional alterations, and can selectively reduce the generation of hydroxyl radicals and peroxynitrite, thereby protecting the cells against oxidant injury. Yokota, Molecular hydrogen protects chrondrocytes from oxidative stress and indirectly alters gene expressions through reducing peroxynitrite derived from nitric oxide. Medical Gas Research 2011.

In an acute rat model in which oxidative stress was induced in the brain by focal FiOischemia-reperfusion (FR), inhaled hydrogen gas markedly suppressed the associated brain injury. Thus it was suggested that administration of hydrogen gas by inhalation may serve as an effective therapy for ischemia-reperfusion, and based on the ability of hydrogen gas to rapidly diffuse across membranes, it can even protect ischemic tissues against oxidative damage. Ohsawa I, et al., Hydrogen acts as a therapeutic antioxidant by selectively reducing cytotoxic oxygen radicals. Nat Med 13: 688-694, 2007. Breathing NO plus hydrogen gas was also found to reduce cardiac injury and augment recovery of the left ventricular function, by elimination of the nitrotyrosine produced by NO inhalation alone. See, e.g., Shinbo, et al., “Breathing nitric oxide plus hydrogen has reduced ischemia-reperfusion injury and nitrotyrosine production in murine heart,” Am J. Physiol Heart Circ Physiol., 305: H542-H550, 2013. In addition, data has indicated that combination therapy with hydrogen gas and NO can effectively attenuate LPS-induced lung inflammation and injury in mice. Liu, et al, “Combination therapy with NO and H₂ in ALI.”

There are several methods to administer hydrogen, such as inhalation of hydrogen gas, aerosol inhalation of a hydrogen-rich solution, drinking hydrogen dissolved in water, injecting hydrogen-rich saline (HRS) and taking a hydrogen bath. Drinking hydrogen solution (saline/pure water/other solutions saturated with hydrogen) may be more practical in daily life and more suitable for daily consumption. Shen, et al., “A review of experimental studies of hydrogen as a new therapeutic agent in emergency and critical care medicine” Medical Gas Research, 2014. Molecular hydrogen diffuses rapidly across cell membranes, reduces reactive oxygen species, including hydroxyl radicals and peroxynitrite, and suppresses oxidative stress-induced injury in several organs with no known toxicity. Fu, et al., Molecular hydrogen is protective against 6-hydroxydopamine-induced nigrostriatal degeneration in a rat model of Parkinson's disease.

### Administering NO

NO can be administered by titration. Titration is a method or process of administering a dose of compound such as NO until a visible or detectable change is achieved.

Any suitable system can be used to deliver NO. NO can be administered by titration. As previously discussed, titration is a method or process of determining the concentration of a dissolved substance in terms of the smallest amount of reagent of known concentration required to bring about a given effect in reaction with a known volume of the test solution.

### Modulating Hormesis

A method of providing NO in a therapeutic setting can include administering exogenous NO to modulate the hormesis characteristics of NO. Hormesis in this instance refers to the temporal and dose dependency related to the stimulatory versus inhibitory response to NO. For example, NO stimulates HIF for 30 minutes at low dose during hypoxia. It becomes inhibitory at high doses and after 30 minutes. This suggests that it would be effective to lower doses 0.1 to 5 ppm for up to 15 to 30 minutes repeated at a intervals rather than high dose continuous delivery, for example. Treatment with exogenous NO may become inhibitory, and therefore, less effective beyond 30 minutes. This suggests that continuous delivery of NO may be less effective than repeated dosing at predefined intervals such as once every hour over a 6, 12, or 24 hour period.

In one embodiment, a nitric oxide delivery system can include a cartridge. A cartridge can include an inlet and an outlet. A cartridge can convert a nitric oxide-releasing agent to nitric oxide (NO). A nitric oxide-releasing agent can include one or more of nitrogen dioxide (NO₂), dinitrogen tetroxide (N₂O₄) or nitrite ions (NO₂⁻). Nitrite ions can be introduced in the form of a nitrite salt, such as sodium nitrite.

A cartridge can include a reducing agent or a combination of reducing agents. A number of reducing agents can be used depending on the activities and properties as determined by a person of skill in the art. In some embodiments, a reducing agent can include a hydroquinone, glutathione, and/or one or more reduced metal salts such as Fe(II), Mo(VI), NaI, Ti(III) or Cr(III), thiols, or NO₂⁻. A reducing agent can include 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid. A reducing agent can be safe (i.e., non-toxic and/or non-caustic) for inhalation by a mammal, for example, a human. A reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above. A reducing agent can be a gas such as hydrogen. A reducing agent can be used dry or wet. For example, a reducing agent can be in solution. A reducing agent can be at different concentrations in a solution. Solutions of the reducing agent can be saturated or unsaturated. While a reducing agent in organic solutions can be used, a reducing agent in an aqueous solution is preferred. A solution including a reducing agent and an alcohol (e.g. methanol, ethanol, propanol, isopropanol, etc.) can also be used.

A cartridge can include a support. A support can be any material that has at least one solid or non-fluid surface (e.g. a gel). It can be advantageous to have a support that has at least one surface with a large surface area. In preferred embodiments, the support can be porous or permeable. One example of a support can be surface-active material, for example, a material with a large surface area that is capable of retaining water or absorbing moisture. Specific examples of surface active materials can include silica gel or cotton. The term “surface-active material” denotes that the material supports an active agent on its surface.

A support can include a reducing agent. Said another way, a reducing agent can be part of a support. For example, a reducing agent can be present on a surface of a support. One way this can be achieved can be to coat a support, at least in part, with a reducing agent. In some cases, a system can be coated with a solution including a reducing agent. Preferably, a system can employ a surface-active material coated with an aqueous solution of antioxidant as a simple and effective mechanism for making the conversion. Generation of NO from a nitric oxide-releasing agent performed using a support with a reducing agent can be the most effective method, but a reducing agent alone can also be used to convert nitric oxide-releasing agent to NO.

In some circumstances, a support can be a matrix or a polymer, more specifically, a hydrophilic polymer. A support can be mixed with a solution of the reducing agent. The solution of reducing agent can be stirred and strained with the support and then drained. The moist support-reducing agent mixture can be dried to obtain the proper level of moisture. Following drying, the support-reducing agent mixture may still be moist or may be dried completely. Drying can occur using a heating device, for example, an oven or autoclave, or can occur by air drying.

In general, a nitric oxide-releasing agent can be converted to NO by bringing a gas including the nitric oxide-releasing agent in contact with a reducing agent. In one example, a gas including a nitric oxide-releasing agent can be passed over or through a support including a reducing agent. When the reducing agent is ascorbic acid (i.e. vitamin C), the conversion of nitrogen dioxide to nitric oxide can be quantitative at ambient temperatures.

The generated nitric oxide can be delivered to a mammal, which can be a human. To facilitate delivery of the nitric oxide, a system can include a patient interface. Examples of a patient interface can include a mouth piece, nasal cannula, face mask, fully-sealed face mask or an endotracheal tube. A patient interface can be coupled to a delivery conduit. A delivery conduit can include a ventilator or an anesthesia machine.

Alternatively or additionally, a NO₂ removal receptacle can be inserted just before the attachment of the delivery system to the patient to further enhance safety and help ensure that all traces of the toxic NO₂ have been removed. The NO₂ removal receptacle may be a receptacle used to remove any trace amounts of NO₂. An example is the technology developed by GeNO and includes the use of ascorbic acid on silica gel, certain secondary and tertiary amines that for nitrosamines that are not carcinogenic and other agents. Alternatively, the NO₂ removal receptacle can include heat-activated alumina. A receptacle with heat-activated alumina, such as supplied by Fisher Scientific International, Inc., designated as ASOS-212, of 8-14 sized mesh can be effective at removing low levels of NO₂ from an air or oxygen stream, and yet, can allow NO gas to pass through without loss. Activated alumina, and other high surface area materials like it, can be used to scrub NO₂ from a NO inhalation line.

In another example, a cartridge can be used to generate NO for therapeutic gas delivery. Because of the effectiveness of a cartridge in converting nitric oxide-releasing agents to NO, nitrogen dioxide (gaseous or liquid) or dinitrogen tetroxide can be used as the source of the NO. When nitrogen dioxide or dinitrogen tetroxide is used as a source for generation of NO, there may be no need for a pressurized gas bottle to provide NO gas to the delivery system. By eliminating the need for a pressurized gas bottle to provide NO, the delivery system may be simplified as compared with a conventional apparatus that is used to deliver NO gas to a patient from a pressurized gas bottle of NO gas. A NO delivery system that does not use pressurized gas bottles may be more portable than conventional systems that rely on pressurized gas bottles.

In some delivery systems, the amount of nitric oxide-releasing agent in a gas can be approximately equivalent to the amount of nitric oxide to be delivered to a patient. For example, if a therapeutic dose of 20 ppm of nitric oxide is to be delivered to a patient, a gas including 20 ppm of a nitric oxide-releasing agent (e.g., NO₂) can be released from a gas bottle or a diffusion tube. The gas including 20 ppm of a nitric oxide-releasing agent can be passed through one or more cartridges to convert the 20 ppm of nitric oxide-releasing agent to 20 ppm of nitric oxide for delivery to the patient. However, in other delivery systems, the amount of nitric oxide-releasing agent in a gas can be greater than the amount of nitric oxide to be delivered to a patient. For example, a gas including 800 ppm of a nitric oxide-releasing agent can be released from a gas bottle or a diffusion tube. The gas including 800 ppm of a nitric oxide-releasing agent can be passed through one or more cartridges to convert the 800 ppm of nitric oxide-releasing agent to 800 ppm of nitric oxide. The gas including 800 ppm of nitric oxide can then be diluted in a gas including oxygen (e.g., air) to obtain a gas mixture with 20 ppm of nitric oxide for delivery to a patient. Traditionally, the mixing of a gas including nitric oxide with a gas including oxygen to dilute the concentration of nitric oxide has occurred in a line or tube of the delivery system. The mixing of a gas including nitric oxide with a gas including oxygen can cause problems because nitrogen dioxide can form. To avoid this problem, two approaches have been used. First, the mixing of the gases can be performed in a line or tube immediately prior to the patient interface, to minimize the time nitric oxide is exposed to oxygen, and consequently, reduce the nitrogen dioxide formation. Second, a cartridge can be placed at a position downstream of the point in the line or tubing where the mixing of the gases occurs, in order to convert any nitrogen dioxide formed back to nitric oxide.

While these approaches can minimize the nitrogen dioxide levels in a gas delivered to a patient, these approaches have some drawbacks. Significantly, both of these approaches mix a gas including nitric oxide with a gas including oxygen in a line or tubing of the system. One problem can be that lines and tubing in a gas delivery system can have a limited volume, which can constrain the level of mixing. Further, a gas in lines and tubing of a gas delivery system can experience variations in pressure and flow rates. Variations in pressure and flow rates can lead to an unequal distribution of the amount each gas in a mixture throughout a delivery system. Moreover, variations in pressure and flow rates can lead to variations in the amount of time nitric oxide is exposed to oxygen within a gas mixture. One notable example of this arises with the use of a ventilator, which pulses gas through a delivery system. Because of the variations in pressure, variations in flow rates and/or the limited volume of the lines or tubing where the gases are mixed, a mixture of the gases can be inconsistent, leading to variation in the amount of nitric oxide, nitrogen dioxide, nitric oxide-releasing agent and/or oxygen between any two points in a delivery system.

To address these problems, a mixing chamber can also be used to mix a first gas and a second gas. A first gas can include oxygen; more specifically, a first gas can be air. A second gas can include a nitric oxide-releasing agent and/or nitric oxide. A first gas and a second gas can be mixed within a mixing chamber to form a gas mixture. The mixing can be an active mixing performed by a mixer within a chamber. For example, a mixer can be a moving support. The mixing within a mixing chamber can also be a passive mixing, for example, the result of diffusion.

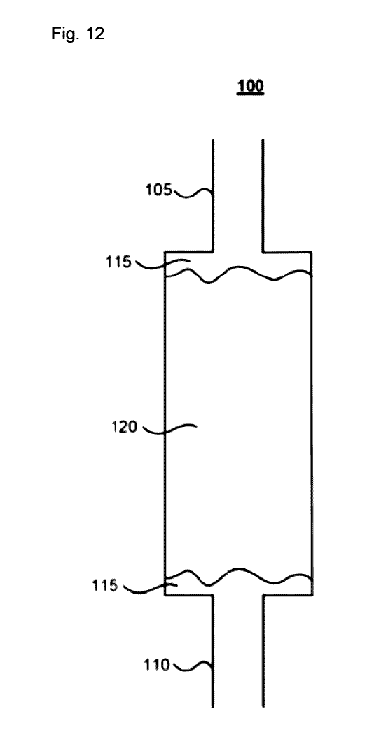

Referring to FIG. 12, this illustrates one embodiment of a cartridge for generating NO by converting a nitric oxide-releasing agent to NO. The cartridge 100 can include an inlet 105 and an outlet 110. The cartridge can include an inlet, an outlet, and a reducing agent. The cartridge can be configured to utilize the whole surface area in converting nitric oxide-releasing agents to NO. The cartridge can have a length, width, and thickness, an outer surface, and an inner surface, and can be substantially cylindrical in shape. The cartridge can have aspect ratio of approximately 2:1, 3:1, 4:1 or 5:1. The length can be, for example, one inch, two inches, three inches, four inches, five inches or six inches. The width can be, for example, 0.5 inch, 1 inch, 1.5 inches, 2 inches, 2.5 inches, or 3 inches. The cartridge can have a cross-section that is a circle, oval, or ellipse. In certain embodiments, opposing sides along the length of the cartridge can be flat. The thickness between the inner and outer surface can be constant, thereby providing a uniform exposure to the reducing agents. The thickness can be approximately 1 mm, 2 mm, 5 mm, 10 mm, 20 mm, 30 mm, or 40 mm for example.

A cartridge can be inserted into and removed from an apparatus, platform or system. Preferably, a cartridge is replaceable in the apparatus, platform or system, and more preferably, a cartridge can be disposable. Screen and glass wool 115 can be located at either or both of the inlet 105 and the outlet 110. The remainder of the cartridge 100 can include a support. In a preferred embodiment, a cartridge 100 can be filled with a surface-active material 120. The surface-active material 120 can be soaked with a saturated solution of antioxidant in water to coat the surface-active material. The screen and glass wool 115 can also be soaked with the saturated solution of antioxidant in water before being inserted into the cartridge 100.

In general, a process for converting a nitric oxide-releasing agent to NO can include passing a gas including a nitric oxide-releasing agent into the inlet 105. The gas can be communicated to the outlet 110 and into contact with a reducing agent. In a preferred embodiment, the gas can be fluidly communicated to the outlet 110 through the surface-active material 120 coated with a reducing agent. As long as the surface-active material remains moist and the reducing agent has not been used up in the conversion, the general process can be effective at converting a nitric oxide-releasing agent to NO at ambient temperature.

The inlet 105 may receive the gas including a nitric oxide-releasing agent from a gas pump that fluidly communicates the gas over a diffusion tube or a permeation cell. The inlet 105 also may receive the gas including a nitric oxide-releasing agent, for example, from a pressurized bottle of a nitric oxide-releasing agent. A pressurized bottle may also be referred to as a tank. The inlet 105 also may receive a gas including a nitric oxide-releasing agent can be NO₂ gas in nitrogen (N₂), air, or oxygen (O₂). A wide variety of flow rates and NO₂ concentrations have been successfully tested, ranging from only a few ml per minute to flow rates of up to 5,000 ml per minute.

The conversion of a nitric oxide-releasing agent to NO can occur over a wide range of concentrations of a nitric oxide-releasing agent. For example, experiments have been carried out at concentrations in air of from about 2 ppm NO₂ to 100 ppm NO₂, and even to over 1000 ppm NO₂. In one example, a cartridge that was approximately 6 inches long and had a diameter of 1.5-inches was packed with silica gel that had first been soaked in a saturated aqueous solution of ascorbic acid. The moist silica gel was prepared using ascorbic acid designated as A.C.S reagent grade 99.1% pure from Aldrich Chemical Company and silica gel from Fischer Scientific International, Inc., designated as S8 32-1, 40 of Grade of 35 to 70 sized mesh. Other sizes of silica gel can also be effective. For example, silica gel having an eighth-inch diameter can also work.

In another example, silica gel was moistened with a saturated solution of ascorbic acid that had been prepared by mixing 35% by weight ascorbic acid in water, stirring, and straining the water/ascorbic acid mixture through the silica gel, followed by draining. The conversion of NO₂ to NO can proceed well when the support including the reducing agent, for example, silica gel coated with ascorbic acid, is moist. In a specific example, a cartridge filled with the wet silica gel/ascorbic acid was able to convert 1000 ppm of NO₂ in air to NO at a flow rate of 150 ml per minute, quantitatively, non-stop for over 12 days.

A cartridge can be used for inhalation therapy. In addition to converting a nitric oxide-releasing agent to nitric oxide to be delivered during inhalation therapy, a cartridge can remove any NO₂ that chemically forms during inhalation therapy (e.g., nitric oxide that is oxidized to form nitrogen dioxide). In one such example, a cartridge can be used as a NO₂ scrubber for NO inhalation therapy that delivers NO from a pressurized bottle source. A cartridge may be used to help ensure that no harmful levels of NO₂ are inadvertently inhaled by the patient.

In addition, a cartridge may be used to supplement or replace some or all of the safety devices used during inhalation therapy in conventional NO inhalation therapy. For example, one type of safety device can warn of the presence of NO₂ in a gas when the concentration of NO₂ exceeds a preset or predetermined limit, usually 1 part per million or greater of NO₂. Such a safety device may be unnecessary when a cartridge is positioned in a NO delivery system just prior to the patient breathing the NO laden gas. A cartridge can convert any NO₂ to NO just prior to the patient breathing the NO laden gas, making a device to warn of the presence of NO₂ in gas unnecessary.

Furthermore, a cartridge placed near the exit of inhalation equipment, gas lines or gas tubing can also reduce or eliminate problems associated with formation of NO₂ that occur due to transit times in the equipment, lines or tubing. As such, use of a cartridge can reduce or eliminate the need to ensure the rapid transit of the gas through the gas plumbing lines that is needed in conventional applications. Also, a cartridge can allow the NO gas to be used with gas balloons to control the total gas flow to the patient.

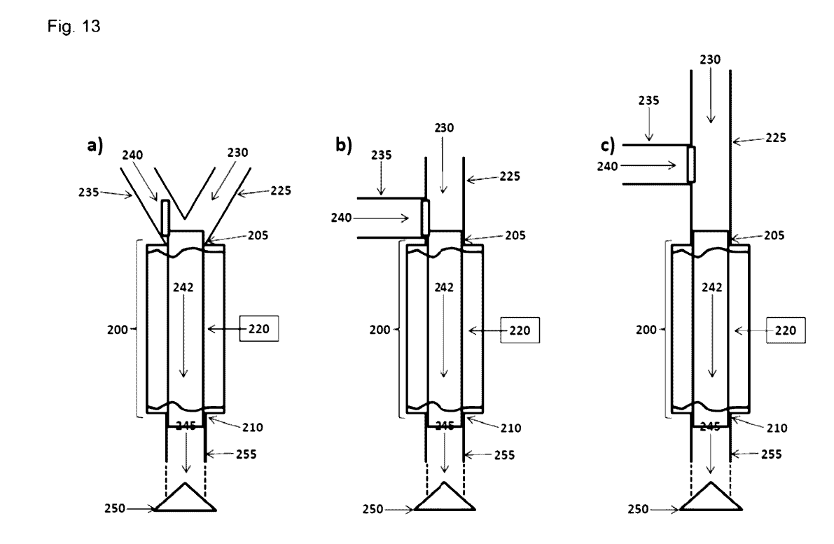

As shown in FIGS. 13 A-C, a cartridge 200 can be coupled to a gas conduit 225. A first gas 230 including oxygen can be communicated through a gas conduit 225 to the cartridge 200. The communication of the first gas through the gas conduit can be continuous or it can be intermittent. For instance, communicating the first gas intermittently can include communicating the first gas through the gas conduit in one or more pulses. Intermittent communication of the first gas through gas conduit can be performed using a gas bag, a pump, a hand pump, an anesthesia machine or a ventilator.

A gas conduit can include a gas source. A gas source can include a gas bottle, a gas tank, a permeation cell or a diffusion tube. Nitric oxide delivery systems including a gas bottle, a gas tank a permeation cell or a diffusion tube are described, for example, in U.S. Pat. Nos. 7,560,076 and 7,618,594, each of which are incorporated by reference in its entirety. Alternatively, a gas source can include a reservoir and restrictor, as described in U.S. patent application Ser. Nos. 12/951,811, 13/017,768 and 13/094,535, each of which is incorporated by reference in its entirety. A gas source can include a pressure vessel, as described in U.S. patent application Ser. No. 13/492,154, which is incorporated by reference in its entirety. A gas conduit can also include one or more additional cartridges. Additional components including one or more sensors for detecting nitric oxide levels, one or more sensors for detecting nitrogen dioxide levels, one or more sensor for detecting oxygen levels, one or more humidifiers, valves, tubing or lines, a pressure regulator, flow regulator, a calibration system and/or filters can also be included in a gas conduit.

A second gas 240 can also be communicated to a cartridge 200. A second gas can be supplied into a gas conduit, as shown in FIGS. 2b and 2c. Preferably, a second gas 240 can be supplied into a gas conduit 225 immediately prior to a cartridge 200, as shown in FIG. 2b. A second gas 240 can be supplied into a gas conduit 225 via a second gas conduit 235, which can join or be coupled to the gas conduit 225. Once a second gas 240 is supplied into a gas conduit 225, both the first gas 230 and the second gas 240 can be communicated in the inlet 205 of a cartridge 200 for mixing. Alternatively, a second gas 240 can be supplied at a cartridge 200, as show in FIG. 2a. For example, a second gas 240 can be supplied directly into the inlet 205 of a cartridge 200.

Once a first gas 230 and a second gas 240 are within a cartridge 200, a first gas 230 and a second gas 240 can mix to form a gas mixture 242 including oxygen and one or more of nitric oxide, a nitric oxide-releasing agent (which can be nitrogen dioxide) and nitrogen dioxide. The gas mixture 242 can contact a reducing agent, which can be on a support 220 within the cartridge. The reducing agent can convert nitric oxide-releasing agent and/or nitrogen dioxide in the gas mixture to nitric oxide.

The gas mixture including nitric oxide 245 can then be delivered to a mammal, most preferably, a human patient. The concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm.

Delivering the gas mixture including nitric oxide from the cartridge 200 to the mammal can include passing the gas mixture through a delivery conduit. A delivery conduit 255 can be located between the cartridge 200 and a patient interface 250. In some embodiments, a delivery conduit 255 can be coupled to the outlet 210 of a cartridge 200 and/or coupled to the patient interface 250. As indicated by the dashed lines in FIGS. 2a, 2b and 2c, a delivery conduit can include additional components, for example, a humidifier or one or more additional cartridges.

Delivery of a gas mixture can include continuously providing the gas mixture to the mammal. When the delivery of the gas mixture includes continuously providing the gas mixture to the mammal, the volume of the cartridge can be greater than the volume of the delivery conduit. The larger volume of the cartridge can help to ensure that the gas mixture is being thoroughly mixed prior to delivery. Generally, more complete mixing can occur as the ratio of the volume of the cartridge to the volume of the delivery conduit increases. A preferable level of mixing can occur when the volume of the cartridge is at least twice the volume of the delivery conduit. The volume of the cartridge can also be at least 1.5 times, at least 3 times, at least 4 times or at least 5 times the volume of the delivery conduit.

When the volume of the cartridge is greater than the volume of the delivery conduit or the volume of gas mixture in the delivery conduit, the gas mixture may not go directly from the cartridge to the mammal, but instead, can be delayed in receptacle or delivery conduit. It is this delay that can provide the time needed to mix the gas so that the NO concentration remains constant within a breath.

This delay can result in the storage of the gas mixture in receptacle. The gas mixture can be stored in the receptacle for a predetermined period of time. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. This can be referred to as “perfect mixing.” For continuous delivery, this can mean that the concentration of nitric oxide in the gas mixture delivered to a mammal remains constant over a period of time (e.g. at least 1 min, at least 2 min, at least 5 min, at least 10 min or at least 30 min). For a concentration to remain constant, the concentration can remain with a range of at most ±10%, at most ±5%, or at most ±2% of a desired concentration for delivery.

Delivery of the gas mixture can include intermittently providing the gas mixture to the mammal. Intermittent delivery of a gas mixture can be the result of intermittent communication of a first or second gas into the system. Said another way, intermittent communication of a first or second gas through a gas conduit can result in an increased area of pressure, which can traverse into a receptacle causing intermittent communication of the gas mixture. Intermittent delivery can be performed using a gas bag, a pump, a hand pump, an anesthesia machine or a ventilator.

The intermittent delivery can include an on-period, when the gas mixture is delivered to a patient, and an off-period, when the gas mixture is not delivered to a patient. Intermittent delivery can include delivering one or more pules of the gas mixture.

An on-period or a pulse can last for a few seconds up to as long as several minutes. In one embodiment, an on-period or a pulse can last for 1, 5, 10, 15, 20, 25, 30, 35, 40, 45, 50, 55 or 60 seconds. In another embodiment, the on-period or a pulse can last for 1, 2, 3, 4 or 5 minutes. In a preferred embodiment, an on-period or a pulse can last for 0.5-10 seconds, most preferably 1-6 seconds.

Intermittent delivery can include a plurality of on-periods or pulses. For example, intermittent delivery can include at least 1, at least 2, at least 5, at least 10, at least 50, at least 100 or at least 1000 on-periods or pulses.

The timing and duration of each on-period or pulse of the gas mixture can be pre-determined. Said another way, the gas mixture can be delivered to a patient in a pre-determined delivery sequence of one or more on-periods or pulses. This can be achieved using an anesthesia machine or a ventilator, for example.

When the delivery of the gas mixture includes intermittently providing the gas mixture to the mammal, the volume of the receptacle can be greater than the volume of the gas mixture in a pulse or on-period. The larger volume of the receptacle can help to ensure that the gas mixture is being thoroughly mixed prior to delivery. Generally, more complete mixing can occur as the ratio of the volume of the receptacle to the volume of the gas mixture in a pulse or on-period delivered to a mammal increases. A preferable level of mixing can occur when the volume of the receptacle is at least twice the volume of the gas mixture in a pulse or on-period. The volume of the receptacle can also be at least 1.5 times, at least 3 times, at least 4 times or at least 5 times the volume of the gas mixture in a pulse or on-period.

When the volume of the receptacle is greater than the volume of the volume of the gas mixture in a pulse or on-period, the gas mixture may not go directly from the receptacle to the mammal, but instead, can be delayed in the receptacle or delivery conduit for one or more pulses or on-periods. It is this delay that can provide the time needed to mix the gas so that the NO concentration remains constant between delivered pulses or on-periods.

In addition to storage as a result of off-periods, the delay caused by the differing volumes can result in the storage of the gas mixture in the receptacle. The gas mixture can be stored in the receptacle for a predetermined period of time. The predetermined period of time can be during or between pulses or on-periods. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. Intermittent delivery an include providing the gas mixture for two or more pulses or on-periods. Using intermittent delivery, the concentration of nitric oxide in each pulse or on-period can vary by less than 10%, by less than 5%, or by less than 2%. In other words, the variation between the concentration of nitric oxide in a first pulse and the concentration of nitric oxide in a second pulse is less than 10% (or less than 5% or 2%) of the concentration of nitric oxide in the first pulse. In another embodiment, using intermittent delivery, the concentration of nitric oxide in each pulse or on-period can vary by less than 10 ppm, less than 5 ppm, less than 2 ppm or less than 1 ppm. Said another way, the difference between the concentration of nitric oxide in a first pulse and the concentration of nitric oxide in a second pulse is less than 10 ppm, less than 5 ppm, less than 2 ppm or less than 1 ppm.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed:

1. A method of improving hemodynamics comprising:
identifying a mammal having or at risk of developing a vascular depletion of nitric oxide due to nitric oxide scavenging by oxyhemoglobin;
positioning a mammal for nitric oxide treatment;
administering nitric oxide for aiding conversion of oxyhemoglobin to methemoglobin;
preventing scavenging effects of oxyhemoglobin; and
introducing the nitric oxide into the mammal's circulation.

2. The method of claim 1 further comprising
mixing a first gas including oxygen and a second gas including a nitric oxide-releasing agent within a receptacle to form a gas mixture, wherein the receptacle includes an inlet, an outlet and a reducing agent; and
contacting the nitric oxide-releasing agent in the gas mixture with the reducing agent to generate nitric oxide.

3. The method of claim 1 further comprising sedating the mammal.

4. The method of claim 3 wherein sedating includes subjecting the mammal to anesthesia.

5. The method of 1, further comprising monitoring oxygen saturation levels.

6. The method of 1, wherein the nitric oxide is inhaled nitric oxide.

7. The method of 1, wherein administering the nitric oxide includes introducing nitric oxide into a respiratory breathing circuit.

8. The method of claim 1 wherein nitric oxide is administered up to 8 ppm.

9. The method of claim 1 wherein nitric oxide is administered up to 0.8 ppm.

10. The method of claim 1 wherein nitric oxide is administered up to 0.08 ppm.

11. The method of claim 1 wherein nitric oxide is administered after a first transfusion.

12. The method of claim 1, wherein the nitric oxide is administered during an exchange transfusion.

13. The method of claim 1, further comprising delivering a hydrogen gas.

14. The method of claim 13, wherein the hydrogen acts to eliminate peroxynitrite, thereby reducing adverse effects of nitric oxide.

15. The method of claim 12, further comprising delivering a subsequent transfusion.

16. The method of claim 1, further comprising culturing red blood cells to detect contamination prior to transfusion.

17. The method of claim 1, wherein nitric oxide is administered in an amount effective to prevent systemic vasoconstriction.

18. The method of claim 1, wherein nitric oxide is administered in an amount effective to prevent pulmonary vasoconstriction.

19. The method of claim 1, wherein the concentration of nitric oxide in the gas mixture delivered is at least 0.1 ppm.

20. The method of claim 1, wherein the concentration of nitric oxide in the gas mixture delivered is up to 5 ppm.

21. The method of claim 1, wherein the method includes exchanging 65 to 85 percent blood volume over a period of 2-12 hours.

22. The method of claim 1, wherein the circulation has estimated circulating blood volume of 80 ml/kg for term babies.

23. The method of claim 1, wherein the circulation has estimated circulating blood volume of 100 ml/kg for preemies.

24. The method of claim 1, further comprising exchanging the same percent blood volume over the same period of time in a transfusion.

25. The method of claim 1, further comprising monitoring calcium (Ca) levels in the mammal during transfusion, and if Ca<0.7 mEq, providing emergency treatment for hypocalcemia at 10 ml CaCl in 50-100 ml D5W given IV over 5 to 10 minutes.

26. The method of claim 1, further comprising monitoring potassium levels in the mammal during transfusion, and if K>6.5, administering 10-15 units IV of regular insulin along with 50 ml D50W, plus/minus 10-20 mg salbutamol by nebulization, and calcium in the presence of malignant cardiac arrhythmias.

27. The method of claim 1, wherein the circulation has elevated circulating cell-free hemoglobin due to acute or chronic hemolysis.

28. The method of claim 1, further comprising administering analgesia.

29. The method of claim 4, wherein level of anesthesia is evaluated continuously.

30. The method of claim 1, wherein the transfusion involves stored blood, greater than 7 days old.

31. The method of claim 1, wherein the transfusion involves fresh blood, no more than 7 days old.

32. The method of claim 1, wherein hydrogen gas is combined with the nitric oxide in a breathing gas.

33. The method of claim 1, wherein the nitric oxide is provided in an amount effective to minimize acute renal injury.

34. The method of claim 1, wherein the nitric oxide is provided in an amount effective to minimize loss of the neuroprotective effect in the brain.

35. The method of claim 1, wherein the nitric oxide is provided in an amount effective to minimize loss of the protective effect in the lungs.

36. The method of claim 1, wherein the nitric oxide is provided in an amount effective to minimize loss of the protective effect in the heart.

37. The method of claim 1, wherein the nitric oxide is provided in an amount effective to minimize loss of the protective effect in the liver.

38. The method of claim 1, wherein the nitric oxide is provided in an amount effective to minimize loss of the protective effect during cardiac injury, hepatic injury pulmonary injury, or a combination of such injuries.

39. The method of claim 1, wherein the nitric oxide is provided in an amount effective to minimize loss of the protective effect during preeclampsia and hemolysis.

40. The method of claim 1, wherein the nitric oxide is provided in an amount effective to minimize loss of the protective effect during disseminated intravascular coagulopathy (DIC).

41. The method of claim 1, wherein the nitric oxide is provided in an amount effective to minimize loss of the protective effect during transplantation or organ preservation during support with mechanical circulatory support devices.

42. The method of claim 41, wherein the support devices include left, right, or biventricular assist devices, during extracorporeal membrane oxygenation (ECMO), and cardiopulmonary bypass procedures.

43. The method of claim 1, wherein the nitric oxide is provided in an effective amount to minimize hemolysis during sepsis.

44. A system for improving hemodynamics comprising
a table for positioning a mammal to receive nitric oxide treatment;
a monitor configured to detect oxygen saturation levels;
a device for administering nitric oxide in an amount and frequency effective to convert oxyhemoglobin to methemoglobin in the mammal's circulation and prevent scavenging effects of oxyhemoglobin, the device including a cartridge to convert nitric oxide-releasing agents to NO, the cartridge including an inlet, an outlet, and a reducing agent.

45. The system s of claim 44, further comprising a sedation source.

46. The system of claim 45, wherein the sedation source includes anesthesia.

47. The system of claim 44, further comprising an analgesia source.

48. The system of claim 44, wherein the cartridge is configured to utilize the whole surface area in converting nitric oxide-releasing agents to NO.

49. The system of claim 44, wherein the reducing agent is ascorbic acid.

50. The system of claim 44, further comprising a transfusion device.

51. The method of claim 1, further comprising administering exogenous NO to modulate the hormesis characteristics of NO.

52. The method of claim 1, wherein the nitric oxide is administered to neonates.

53. The method of claim 1, wherein the nitric oxide is administered to pediatric patients.

54. The method of claim 1, wherein the nitric oxide is administered to adults.

55. The method of claim 1, wherein the nitric oxide is provided in an amount effective to minimize loss of the protective effect during transplantation and organ preservation, during support with mechanical circulatory support devices.

56. The method of claim 41, wherein the support devices include left, right, and biventricular assist devices, during extracorporeal membrane oxygenation (ECMO), and cardiopulmonary bypass procedures.
